(19) United States
(12) Patent Application Publication
(10) Pub. No.: US 2021/0275160 A1
(43) Pub. Date: Sep. 9, 2021

(54) SOFT ORTHOPEDIC ANCHOR ASSEMBLY AND INSTRUMENTS FOR INSERTING SAME

(71) Applicant: Embody, Inc., Norfolk, VA (US)

(72) Inventors: Douglas Snell, Overland Park, KS (US); Mark G. Messmann, Larwill, IN (US); Isaac Running, Bozeman, MT (US)

(21) Appl. No.: 17/173,938

(22) Filed: Feb. 11, 2021

Related U.S. Application Data
(60) Provisional application No. 62/975,038, filed on Feb. 11, 2020.
(60) Provisional application No. 62/975,013, filed on Feb. 11, 2020.
(60) Provisional application No. 62/975,052, filed on Feb. 11, 2020.

(51) Int. Cl. A61B 17/04; A61B 17/06; A61L 17/00

(52) U.S. Cl. CPC A61B 17/0401; A61B 17/06166; A61B 2017/0458; A61B 2017/042; A61B 2017/0409; A61L 17/00

(57) ABSTRACT

An orthopedic anchor assembly may include an elongate, relatively soft orthopedic anchor; and a suture strand extending from a first end of the orthopedic anchor through a sidewall of the orthopedic anchor and internally through the orthopedic anchor to a second end of the orthopedic anchor. The suture strand may further extend through the sidewall of the orthopedic anchor at the second end of the orthopedic anchor and extend back to the first end of the orthopedic anchor externally to the orthopedic anchor.

### CROSS-REFERENCE TO RELATED APPLICATIONS

This application claims the benefit of U.S. Provisional Patent Application No. 62/975,013, filed on Feb. 11, 2020, and titled “Soft Orthopedic Anchor Assembly;” U.S. Provisional Patent Application Number 62/975,038, filed on Feb. 11, 2020, and titled “Soft Orthopedic Anchor Assembly and Instruments for Inserting Same;” and U.S. Provisional Patent Application No. 62/975,052, filed on Feb. 11, 2020, and titled “Bone Anchor Insertion Tool,” each of which is incorporated herein by reference in its entirety.

### BACKGROUND OF THE INVENTION

The present invention relates generally to an assembly for soft orthopedic anchors for orthopedic surgical repairs and, more specifically, to a particular threading of braided suture through soft orthopedic anchors.

Tissue anchors may use sutures to link native tissue with bone or with graft material. It can be difficult, in some cases, to tighten sutures threaded through a soft anchor. In addition, it can be difficult to utilize multiple anchors in close proximity to one another.

The present disclosure is directed to addressing one or more of the issues discussed above.

### SUMMARY OF THE INVENTION

In one aspect, the present disclosure is directed to an orthopedic anchor assembly. The orthopedic anchor assembly may include an elongate, relatively soft orthopedic anchor. The suture strand may extend from a first end of the orthopedic anchor and internally through the orthopedic anchor to a second end of the orthopedic anchor. The suture strand may further extend through the sidewall of the orthopedic anchor at the second end of the orthopedic anchor and extending back to the first end of the orthopedic anchor externally to the orthopedic anchor.

In another aspect, the present disclosure is directed to a multi-anchor system. The system may include a first elongate, relatively soft orthopedic anchor and a second elongate, relatively soft orthopedic anchor. The system may further include a suture strand extending from a first end of the first orthopedic anchor through a sidewall of the first orthopedic anchor and internally through the first orthopedic anchor to a second end of the first orthopedic anchor; the suture strand further extending through the sidewall of the first orthopedic anchor at the second end of the first orthopedic anchor; the suture strand extending from the second end of the first orthopedic anchor to a first end of the second orthopedic anchor; the suture strand further extending through the second orthopedic anchor to a second end of the second orthopedic anchor; the suture strand extending from the second end of the second orthopedic anchor back to the first end of the first orthopedic anchor; the suture strand extending into itself proximate the first end of the first orthopedic anchor and extending coaxially within a length of the suture strand through the first orthopedic anchor and exiting the length of suture strand proximate the second end of the first orthopedic anchor.

In another aspect, the present disclosure is directed to an insertion tool for an orthopedic anchor. The insertion tool may include a tissue piercing rod received within a first lumen; an anchor delivery rod received within a second lumen parallel to the first lumen; and a distal tip including an exit lumen in communication with both the first lumen and the second lumen. The exit lumen may be configured to alternately receive the tissue piercing rod and the anchor delivery rod.

Other systems, methods, features, and advantages of the invention will be, or will become, apparent to one of ordinary skill in the art upon examination of the following figures and detailed description. It is intended that all such additional systems, methods, features, and advantages be included within this description and this summary, be within the scope of the invention, and be protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like reference numerals designate corresponding parts throughout the different views.

FIG. 1 is a schematic view of a step in a procedure for repairing a rotator cuff tendon, according to an embodiment;

FIG. 2 is a schematic view of a step of applying a graft to a portion of a rotator cuff tendon to facilitate healing, according to an embodiment;

FIG. 3 is a schematic view of an anchor assembly including a soft anchor having a suture strand threaded therethrough according to a disclosed embodiment;

FIG. 4 is a schematic view of the anchor assembly of FIG. 3 showing a first tightening step of pulling a suture loop;

FIG. 5 is a schematic view of the anchor assembly of FIG. 3 showing a second tightening step of pulling a free end of the suture strand forming a drawstring;

FIG. 6 is a schematic partial cross-sectional view of the insertion tool of FIG. 1 piercing through soft tissue and bone;

FIG. 7 is a schematic partial cross-sectional view of the insertion tool of FIG. 1 being withdrawn from the bone and leaving a bone anchor behind; and

FIG. 8 is a schematic perspective view of a hitching post configuration of pinning down sutures extending from a sheet of tendon/ligament graft material.

FIG. 9 is a schematic view of a multi-anchor system according to an exemplary embodiment;

FIG. 10 is a schematic view of the multi-anchor system of FIG. 9 showing a first tightening step of pulling a first suture loop;

FIG. 11 is a schematic view of the multi-anchor system of FIG. 9 showing a second tightening step of pulling a second suture loop;

FIG. 12 is a schematic view of the multi-anchor system of FIG. 9 showing a third tightening step of pulling a large suture loop;

FIG. 13 is a schematic view of the multi-anchor system of FIG. 9 showing a fourth tightening step of pulling a free end of the suture strand forming a drawstring;

FIG. 14 is a schematic view of a step of anchoring a graft to underlying tissue, according to an embodiment;

FIG. 15 is a schematic view of a step of anchoring a graft to underlying tissue, according to an embodiment;

FIG. 16 is a schematic view of a graft that has been anchored to underlying tissue, according to an embodiment;

FIG. 17 is a schematic view of an orthopedic anchor insertion tool according to an exemplary embodiment;

FIG. 18 is a schematic enlarged view of a distal end of the insertion tool shown in FIG. 17;

FIG. 19 is a schematic further enlarged view of the distal end of the insertion tool;

FIG. 20 is a schematic view of a proximal end of the insertion tool;

FIG. 21 is a schematic side view of the insertion tool;

FIG. 22 is a schematic side view of the insertion tool with the tissue piercing rod deployed;

FIG. 23 is a schematic side view of the insertion tool with the anchor delivery rod partially deployed;

FIG. 24 is a schematic enlarged side view of the distal end of the insertion tool with the anchor delivery rod fully deployed and delivering a soft orthopedic anchor;

FIG. 25 is a schematic enlarged view of the insertion tool inserted into tissue and piercing bone with the tissue piercing rod;

FIG. 26 is a schematic enlarged view of the insertion tool inserted into tissue and delivering a soft orthopedic anchor into the hole created by the tissue piercing rod; and

FIGS. 27-34 are schematic illustrations of an orthopedic anchor insertion tool according to another exemplary embodiment.

### DETAILED DESCRIPTION

As used herein, the term “fixedly attached” shall refer to two components joined in a manner such that the components may not be readily separated (for example, without destroying one or both components). The term “removably attached” shall refer to components that are attached to one another in a readily separable manner (for example, with fasteners, such as bolts, screws, etc.).

The embodiments provide an anchoring system that can be used to secure a graft in place over underlying tissue (such as tendons and/or bones) within the body. For example, the anchoring system can be used to secure the graft over a rotator cuff tendon (e.g., the supraspinatus tendon) and/or part of the humerus bone. The anchoring system includes an anchoring device and a deployment device (or instrument) that is used to insert portions of the anchoring device through a graft and underlying tissue.

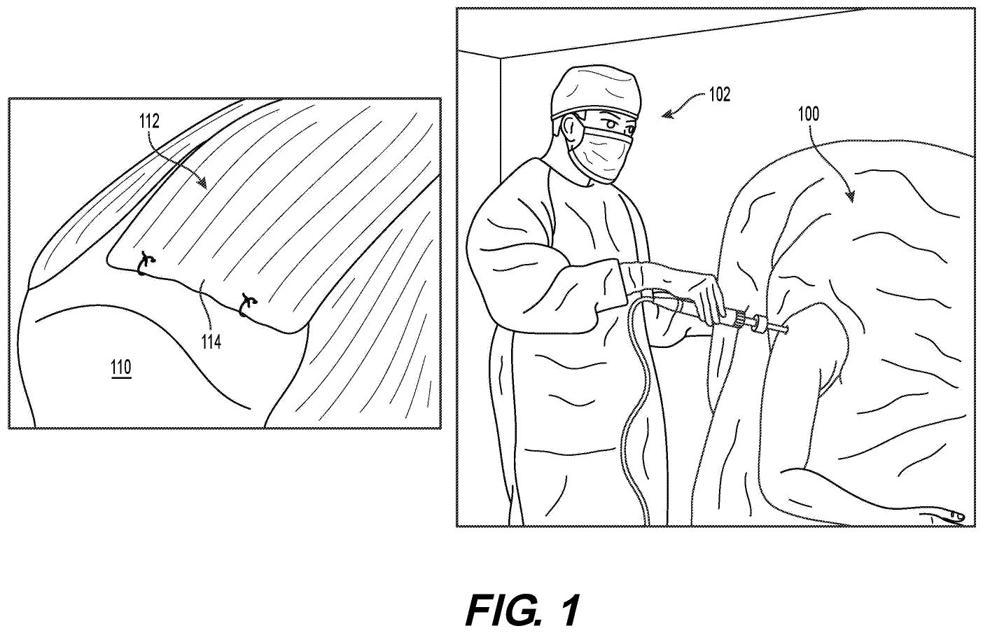

FIG. 1 is a schematic view illustrating a surgical procedure to repair a tendon in a patient's shoulder. Specifically, a patient 100 is undergoing arthroscopic surgery that is performed by surgeon 102. Also shown in FIG. 1 is an enlarged view of a portion of humerus 110 and rotator cuff tendons 112. In the present example, surgeon 102 has recently applied anchors and sutures to secure supraspinatus tendon 114 to humerus 110.

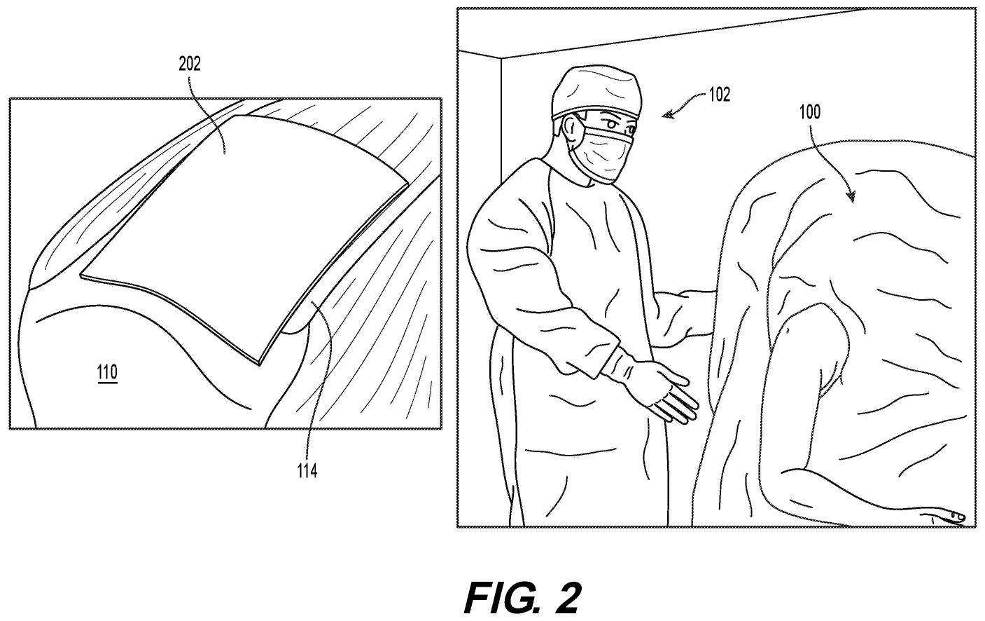

Once the tendon has been sufficiently repaired, surgeon 102 may insert a graft through an incision (possibly using another device to facilitate insertion). The graft can then be placed over the repaired tendon and/or portion of the underlying bone in order to facilitate healing. As an example, FIG. 2 shows a schematic view of a graft 202 that has been applied over the recently repaired tendon 114 as well as over a portion of humerus 110.

Although the exemplary embodiment depicts a procedure in which a tendon is first secured to the bone using sutures and anchors, in other embodiments a graft can be applied to one or more tendons without first reattaching a tendon. For example, grafts could be applied to tendons that have only partial tears.

Once graft 202 has been placed over the repaired tendon, one or more sutures or anchors are required to hold graft 202 in place. The present embodiments disclose anchoring systems that can be used to hold a graft in place.

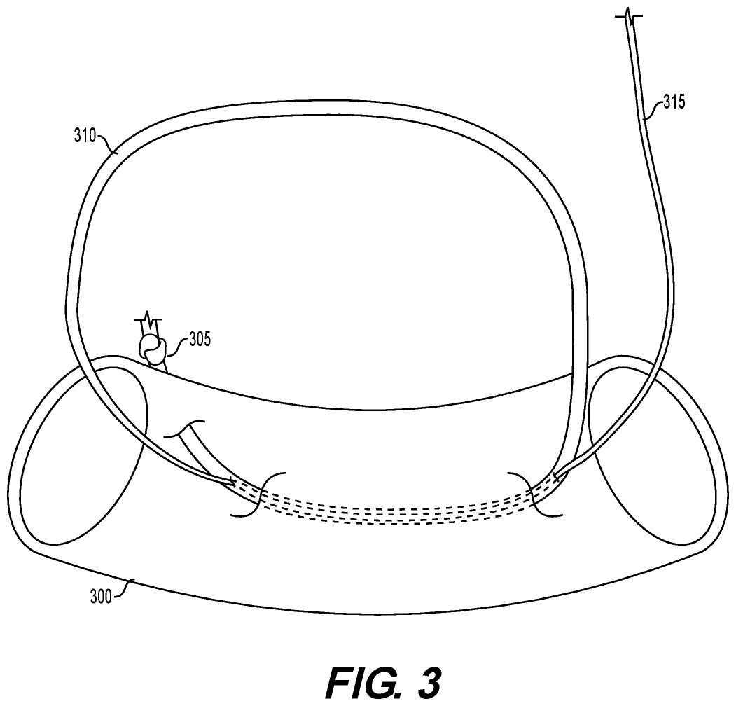

FIG. 3 is a schematic view of an anchor assembly including a soft anchor having a suture strand threaded therethrough according to a disclosed embodiment. In some embodiments, the soft anchor may be an “all suture anchor.” FIG. 3 shows a soft anchor 300 with a suture strand threaded through it in a particular configuration. In particular, the suture strand has a knot 305 tied in in, then extends through the sidewall of anchor 300, then back out of the sidewall and back into the sidewall. Then the suture extends internally to the anchor 300 along the length of the anchor from a first end of the anchor (near knot 305) to a second end of anchor 300. At the second end of the anchor, the suture extends out through the sidewall of anchor 300 again, and back around to the first end of the anchor, forming a suture loop 310. Then, the suture strand extends into itself (note: braided sutures are often hollow). The strand then extends coaxially through itself through the length of anchor 300 and back out of itself at the second end of anchor 300 to a free end 315 that forms a drawstring.

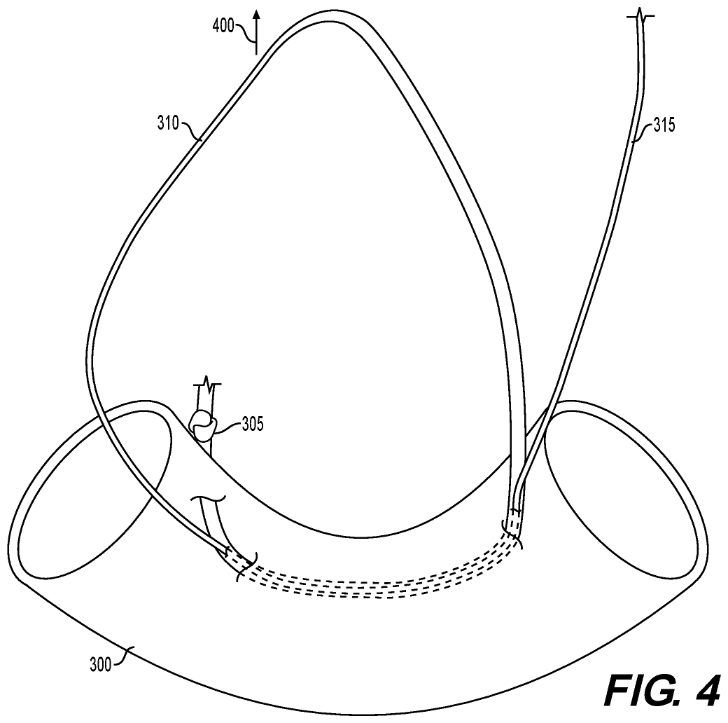

FIG. 4 is a schematic view of the anchor assembly of FIG. 3 showing a first tightening step of pulling a suture loop. In particular, as illustrated by an arrow 400, by pulling on suture loop 310, the suture strand may be partially tightened.

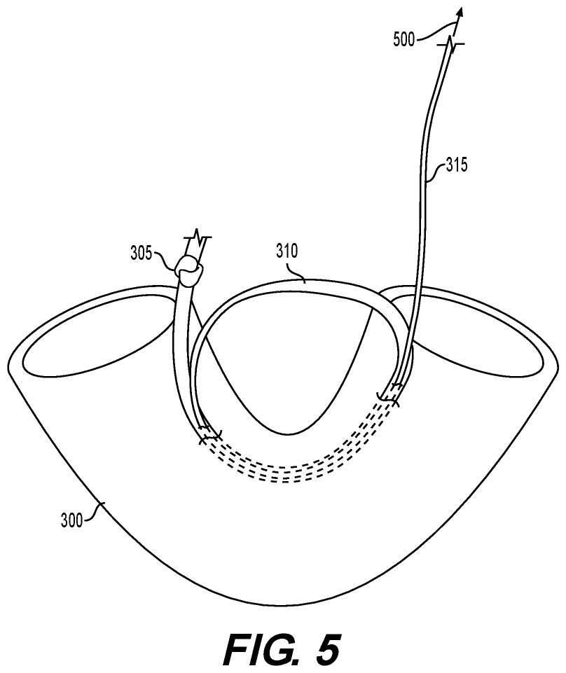

FIG. 5 is a schematic view of the anchor assembly of FIG. 3 showing a second tightening step of pulling a free end of the suture strand forming a drawstring. As shown in FIG. 5 by an arrow 500, subsequently pulling on drawstring 315 may further tighten the suture strand.

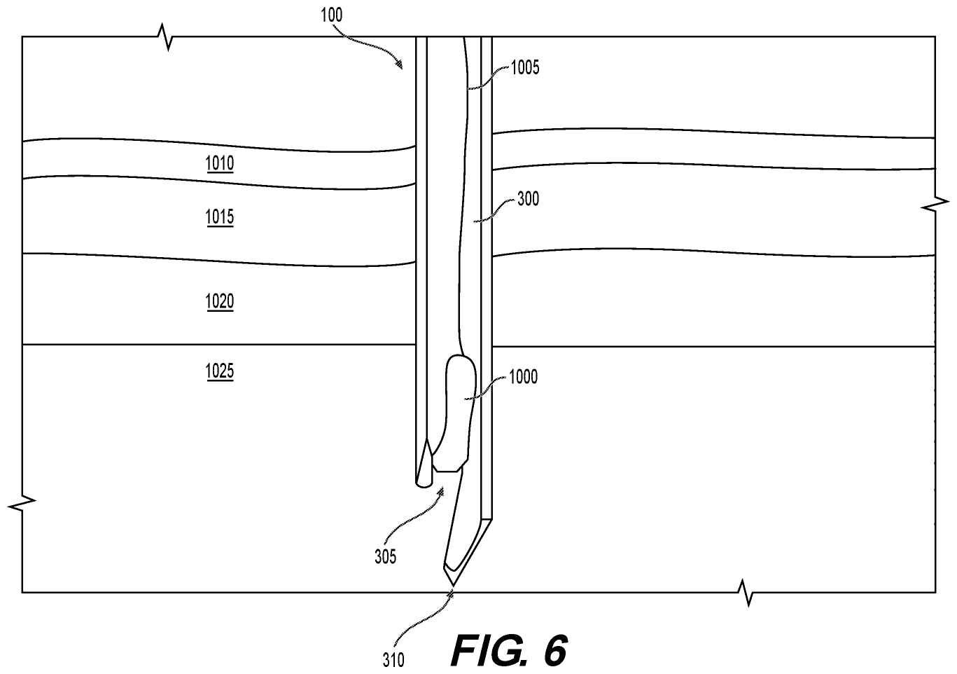

In some embodiments, a single anchor assembly having the configuration described above may be used. In some cases, more than one such anchor assembly may be used. An insertion tool may be configured for inserting one such anchor assembly at a time. Once the anchor is inserted into the bone, another suture may be passed through the suture loop. By tightening the suture strand through the anchor, the suture extending through the suture loop may be pinned in place. FIGS. 6-8 illustrate this functionality of the anchor assembly discussed above.

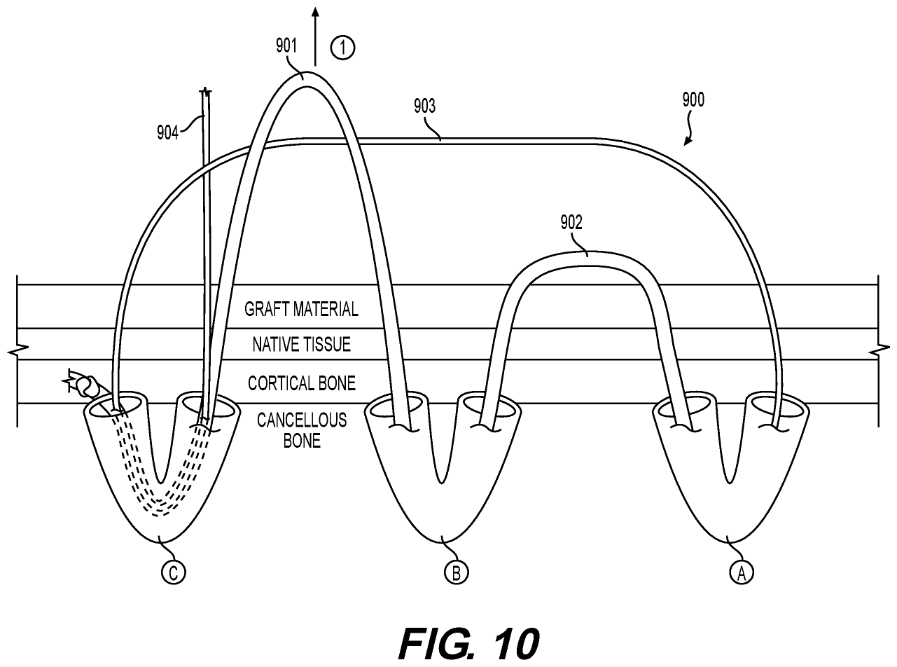

FIG. 6 is a schematic partial cross-sectional view of an insertion tool piercing through soft tissue and bone to insert a bone anchor. As shown in FIG. 6, a bone anchor 1000 may be disposed through slot 305 in an insertion tool 100 and along first rounded groove 300 and second rounded groove 400 (not shown in FIG. 6). The rounded grooves enable the length of bone anchor 1000 to fit alongside the shaft of tool 100 as it is passed through soft tissue and into bone. For example, as shown in FIG. 10, tool 100 may be driven through skin 1010, other soft tissues 1015, cortical bone 1020, and into cancellous bone 1025. It will be understood that, in some cases, insertion tool 100 may be used to push anchors through tissue grafts, such as tendon/ligament grafts.

As also shown in FIG. 6, a suture 1005 may extend from bone anchor 1000 back out of the path of insertion. Suture 1005 may pass through a cavity provided by one of the rounded grooves (300, 400).

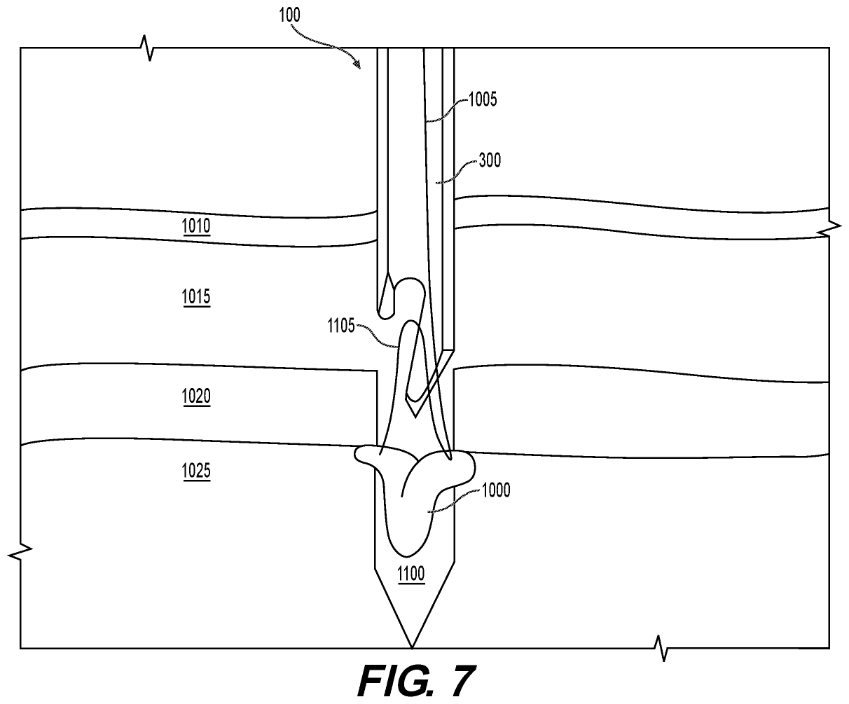

FIG. 7 is a schematic partial cross-sectional view of the insertion tool of FIG. 1 being withdrawn from the bone and leaving a bone anchor behind. As shown in FIG. 7, insertion tool 100 has been completely withdrawn from the cancellous bone 1025 and nearly completely withdrawn from the cortical bone 1020, leaving behind a hole 1100. As further shown in FIG. 7, anchor 1000 is left behind in the region of cancellous bone 1025 below the cortical bone 1020. In order for this to happen, anchor 1000 may be formed of a relatively soft material, including various soft/flexible polymers. In some embodiments, the anchor may be made of a multifilament open braid, (e.g., polyester or polyethylene, nylon, resorbable materials, or collagen) open braid. In some cases, the anchor may be formed of silicone or resorbable tubing.

When retracting insertion tool 100, anchor 1000 catches on the edges of the hole in the cortical bone 1020 and mushrooms such that the anchor becomes wider across than the diameter of the hole in the cortical bone 1020. Anchor 1000 is able to expand under cortical bone 1020 because of the porosity and sponginess of the cancellous bone 1025.

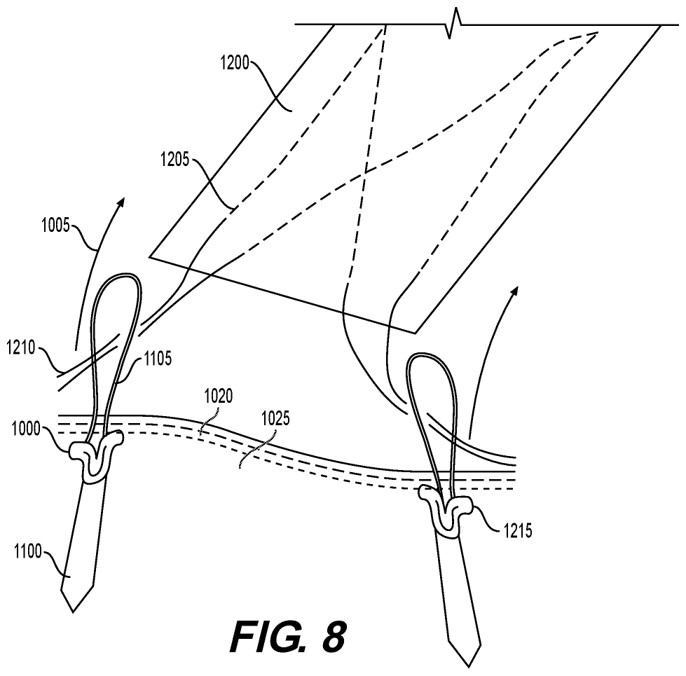

As shown in FIG. 7, in some embodiments, a suture may be threaded through anchor 1000. The threading of the suture through anchor 1000 may be performed in any suitable manner disclosed herein. For example, the suture may be formed into a suture loop 1105, which may extend from anchor 1000 back out of the hole in the cancellous bone 1020. In addition, a free end of the suture may form a drawstring 1005. By pulling on drawstring 1005, suture loop 1105 may be tightened. The application of the suture loop 1105 and drawstring 1005 is shown in further detail in FIG. 8.

In some embodiments, the bone anchors with suture loops and drawstrings may be utilized in a hitching post fashion, where the suture loops are tightened to pin down other sutures extending from a tissue graft, such as a tendon/ligament graft.

FIG. 8 is a schematic perspective view of a hitching post/cargo net configuration of pinning down sutures extending from a sheet of tendon/ligament graft material. As shown in FIG. 8, a tendon/ligament graft 1200 may be sutured to a natural tendon/ligament, for example, using sutures 1205. In order to draw graft 1200 tight, the corners of graft 1200 may be pulled tight by pulling on sutures 1205 and pinning sutures 1205 proximate to the corners of graft 1200. For example, FIG. 8 shows bone anchor 1000 inserted below cortical bone 1020 in cancellous bone 1025, with suture loop 1105 protruding from cortical bone 1020. A free end 1210 of suture 1205 is drawn through suture loop 1105. Then drawstring 1005 is pulled to tighten suture loop 1105 in order to pin down free end 1210 of suture 1205. The same may be done at multiple regions/corners of graft 1200. For example, a second anchor 1215 is shown at a second corner of graft 1200.

In some embodiments, the threading of the anchor assembly described above may be utilized for a multi-anchor system. For example, a multi-anchor system may include a series of anchors tied together with a suture strand in a daisy chain configuration, wherein at least one of the anchors has the suture strand threaded through it in the manner discussed above.

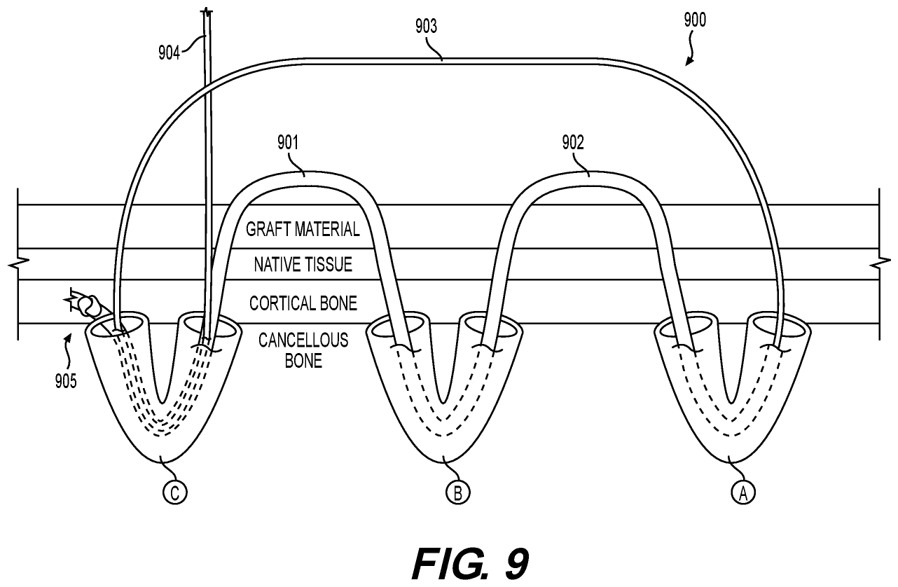

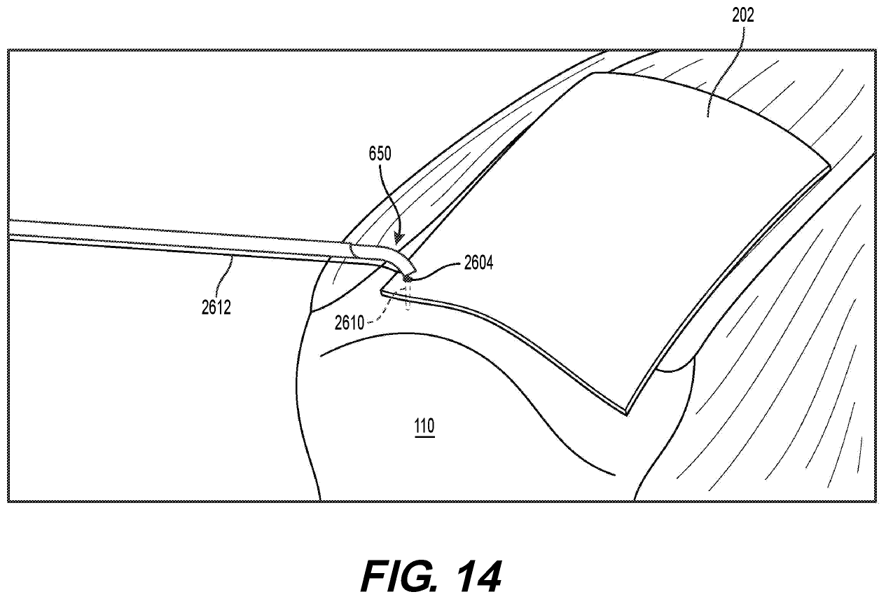

FIG. 9 is a schematic view of a multi-anchor system according to an exemplary embodiment. As shown in FIG. 9, a multi-anchor system 900 may be utilized to secure graft material to native tissue such as tendons, ligaments, and/or bone. As shown in FIG. 9, the anchors may be inserted through graft material, native tissue, cortical bone, and down into cancellous bone. The rigidity of cortical bone may prevent the soft anchors from pulling out through the hole formed in the cortical bone. It will be noted that sutures extending from both ends of the same anchor will pass through the same hole in the tissue/bone. However, for purposes of illustration, the two ends of each suture are shown separately for clarity of the disclosed structures. As shown in FIG. 9, three anchors are inserted. However, it will be understood that system 900 may include any suitable number of anchors. System 900 may include as few as two anchors, and as many anchors as can practically be tightened with the suture strand threaded as disclosed. Exemplary embodiments may include four anchors per system (see FIGS. 14-16).

As shown in FIG. 9, a first anchor A, a second anchor B, and a third anchor C may be inserted through graft material and native tissue. In some embodiments, the anchors may be inserted in that order.

A single suture strand may be threaded through all anchors in the system. A first free end 905 of the suture strand is fixed with a knot. A second free end of the suture strand extends as a drawstring 904.

Only a single anchor of the system will have the suture strand threaded through itself. This coaxial threading of the suture strand provides fixation when cinched down against the graft. In the present embodiment, third anchor C has the coaxial suture-in-suture configuration. Anchors A and B simply have the suture strand pass through each anchor once.

As configured, when all three anchors are inserted, three suture loops are formed. A first suture loop 901 is formed between third anchor C and second anchor B. A second suture loop 902 is formed between second anchor B and first anchor A. And, a third suture loop 903 is formed extending from first anchor A to third anchor C.

FIGS. 10-13 illustrate the four steps of tightening the suture strand to cinch down the anchor system 900.

FIG. 10 is a schematic view of the multi-anchor system of FIG. 9 showing a first tightening step of pulling a first suture loop. As shown in FIG. 10, first suture loop 901 may be pulled (e.g., with a surgical probe), as indicated by an arrow 1. This may tighten the suture strand extending through third anchor C.

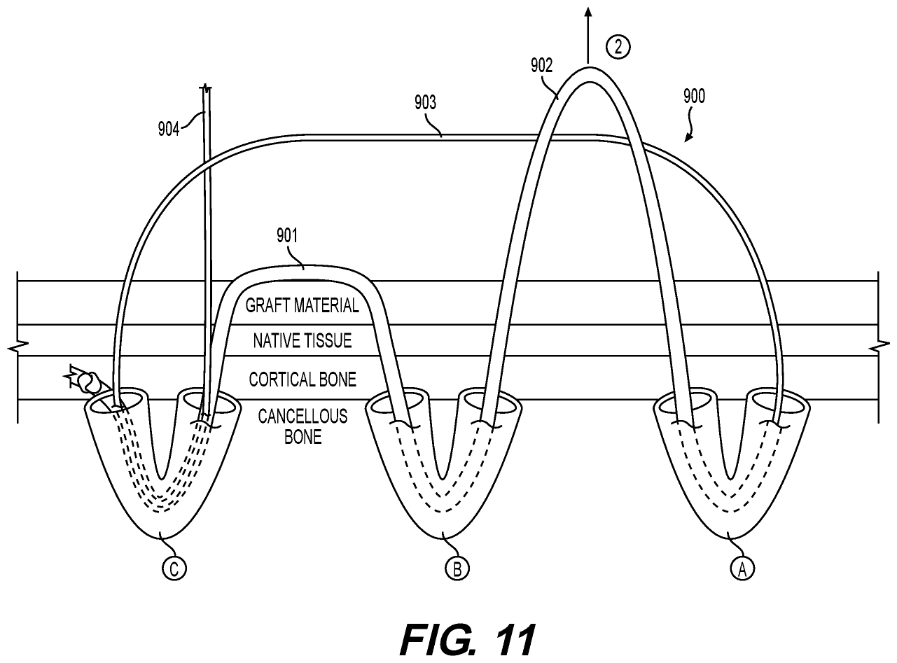

FIG. 11 is a schematic view of the multi-anchor system of FIG. 9 showing a second tightening step of pulling a second suture loop. As shown in FIG. 11, second suture loop 902 may be pulled as indicated by an arrow 2. This may tighten first suture loop 901 against the graft material.

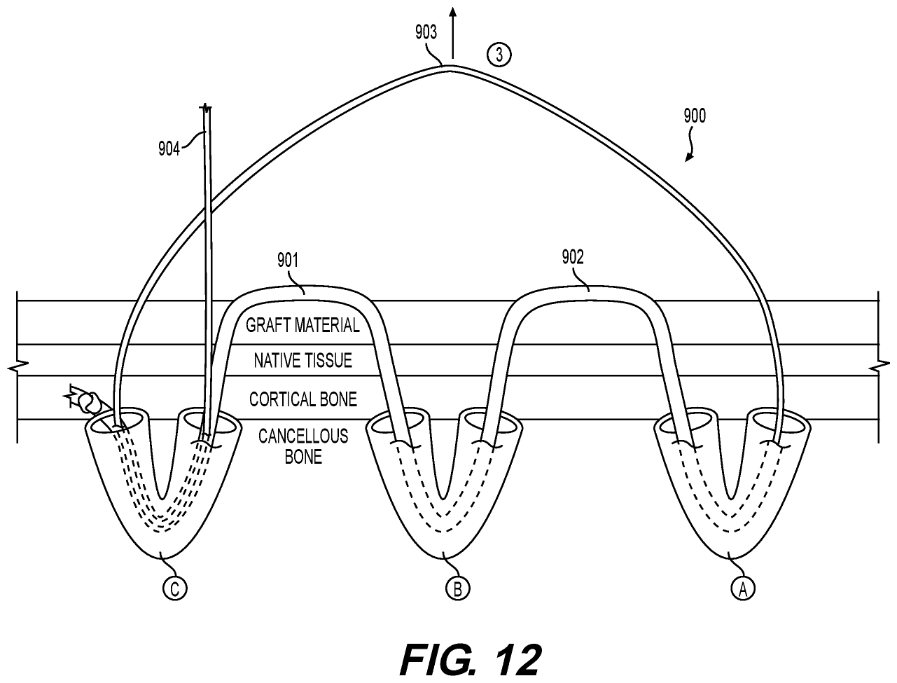

FIG. 12 is a schematic view of the multi-anchor system of FIG. 9 showing a third tightening step of pulling a large suture loop. As shown in FIG. 12, third suture loop 903 (i.e., the large loop) may be pulled, as indicated by an arrow 3. This may tighten second suture loop 902 against the graft material.

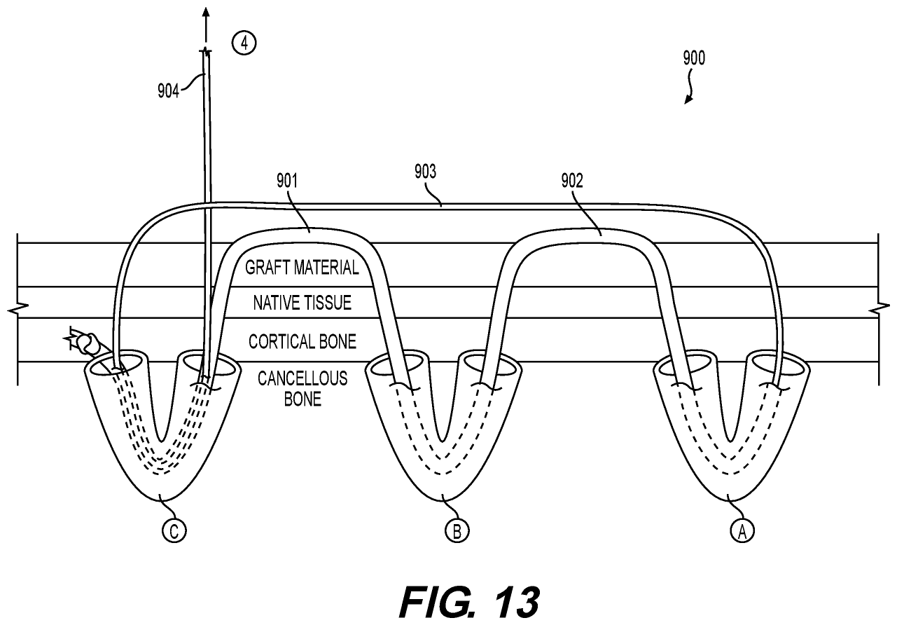

FIG. 13 is a schematic view of the multi-anchor system of FIG. 9 showing a fourth tightening step of pulling a free end of the suture strand forming a drawstring. Finally, as shown in FIG. 13, drawstring 904 may be pulled as indicated by an arrow 4. This may tighten third suture loop 903, balance the tensions in the various suture segments, and cinch the entire system down, pinning the graft material to the native tissue.

FIGS. 14-16 depict schematic views of a graft being anchored to an underlying bone and tendons using the exemplary anchoring system discussed above. As seen in FIG. 14, the tip portion 650 of an anchor deployment device may be positioned at a first location 2604 of a supraspinatus tendon graft 202. At first location 2604, a first anchor 2610 of an anchor system may be inserted through graft 202 and into humerus 110.

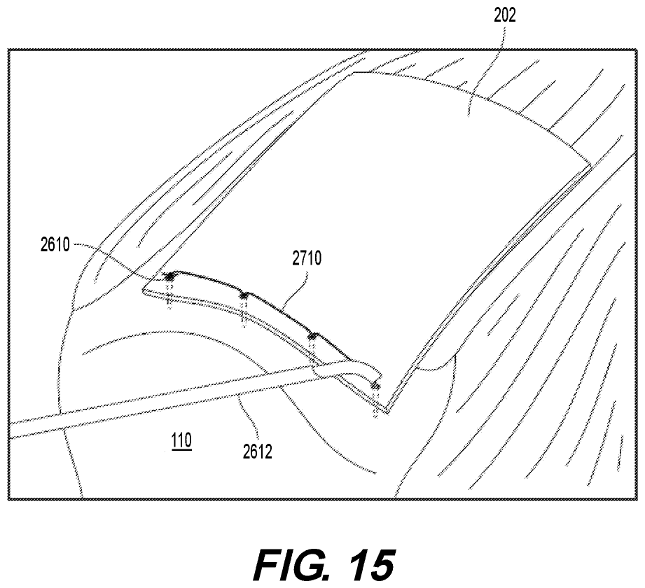

In an exemplary use of the disclosed anchor system, a plurality of anchors of an anchor system may be inserted through graft 202. For example, as shown in FIG. 15, four anchors may be positioned, and tied together via suture strand 2710.

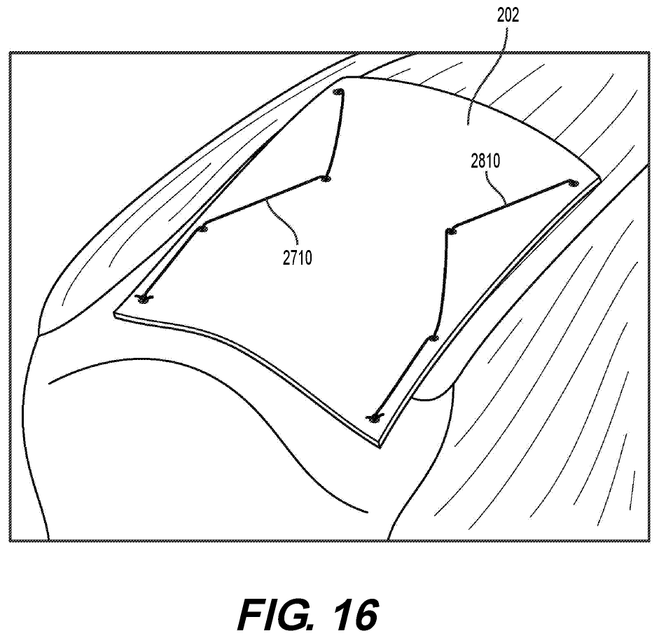

FIG. 15 illustrates the anchoring of a supraspinatus tendon graft along the lateral edge. In some cases, the graft may be anchored to the supraspinatus tendon with the multi-anchor system extending in a medial direction from the lateral edge. For example, as shown in FIG. 16, the anchors may extend medially and may be joined by suture 2710. In some cases, a similar process may be used to lay down a second multi-anchor system along a second edge of graft 202, as shown in FIG. 16. This creates a second anchored suture 2810 along the second edge of graft 202.

In some cases, more than one anchor from the same system may be inserted into the same hole in the bone or other tissue. For example, if the anatomic structure being anchored is relatively narrow, only two holes may be created in the tissue instead of four. Two anchors may be placed in each of the two holes, and the system may be cinched down in a similar fashion as a four-hole, four anchor installation.

The sutures discussed herein may be formed of materials that are biocompatible, and thus suitable for implantation in the body. For example, in some embodiments, the sutures may be formed of absorbable materials such as polyglycolic acid, polylactic acid, monocryl and polydioxanone. Additionally, or alternatively, the sutures may be formed of non-absorbable materials, such as nylon, polyester, polyethylene, PVDF, and polypropylene. In some embodiments, the sutures could be formed of collagen or collagen blended with another polymer. In some embodiments, the sutures may be formed of high strength collagen blended with ultra high molecular weight polyethylene (UHMWPE). Further, in some embodiments, the sutures may be braided. In some cases, the sutures may include any of the braided constructions discussed in Francis et al., U.S. patent application Ser. No. 17/162,568, filed Jan. 29, 2021, and entitled “Braided Surgical Implants,” the entire disclosure of which is incorporated herein by reference.

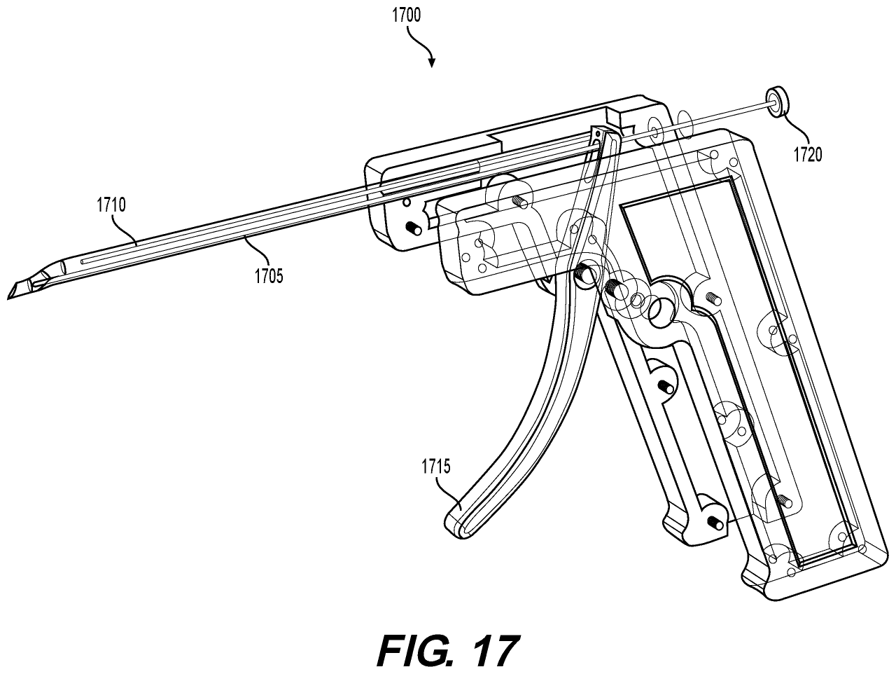

FIG. 17 is a schematic view of an orthopedic anchor insertion tool according to an exemplary embodiment. As shown in FIG. 17, an insertion tool 1700 may include a tissue piercing rod 1705 and an anchor delivery rod 1710. Anchor delivery rod 1710 may be deployed by a trigger type handle 1715. Tissue piercing rod 1705 may be deployed by pressing on a plunger knob 1720.

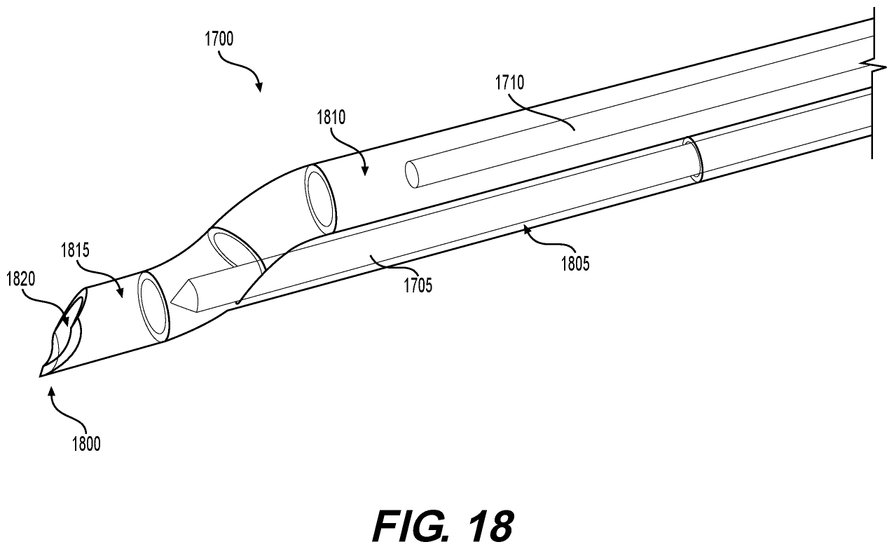

FIG. 18 is a schematic enlarged view of a distal end of the insertion tool shown in FIG. 17. As shown in FIG. 18, a distal tip 1800 of tool 1700 may have a pointed and scalloped end. Such features may facilitate introducing the instrument through soft tissues. The scalloped end may also provide a relief in the tip to permit sutures tying anchors together to remain out of the way when the piercing rod is deployed. Further, in some embodiments, a drilling bit may be utilized instead of the piercing rod. Accordingly, the scalloped end of distal tip 1800 or other relief may be provided in order to provide a place for the sutures tying anchors together to remain while the piercing/drilling rod is deployed. For example, in some embodiments, a cutout or groove may be provided on the inner surface of the tool exit lumen.

As also shown in FIG. 18, tissue piercing rod 1705 may be received within a first lumen 1805. In addition, anchor delivery rod 1710 may be received within a second lumen 1810. Tool 1700 may include a single exit lumen having an exit opening 1820 common to both first lumen 1805 and second lumen 1810. Accordingly, tissue piercing rod 1705 and anchor delivery rod may be alternately deployed through exit lumen 1815 and exit opening 1820. This configuration may ensure that, once a hole is made in the tissue by tissue piercing rod 1705, anchor delivery rod 1710 is driven in the same location as the hole made by tissue piercing rod 1705 (i.e., since the two rods come out of the same opening in the distal end of the tool).

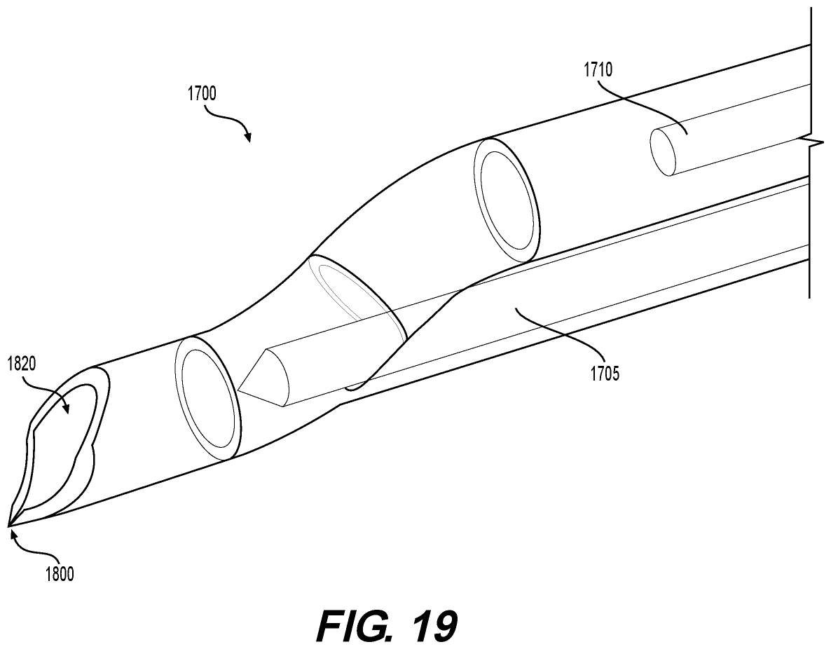

FIG. 19 is a schematic further enlarged view of the distal end of the insertion tool.

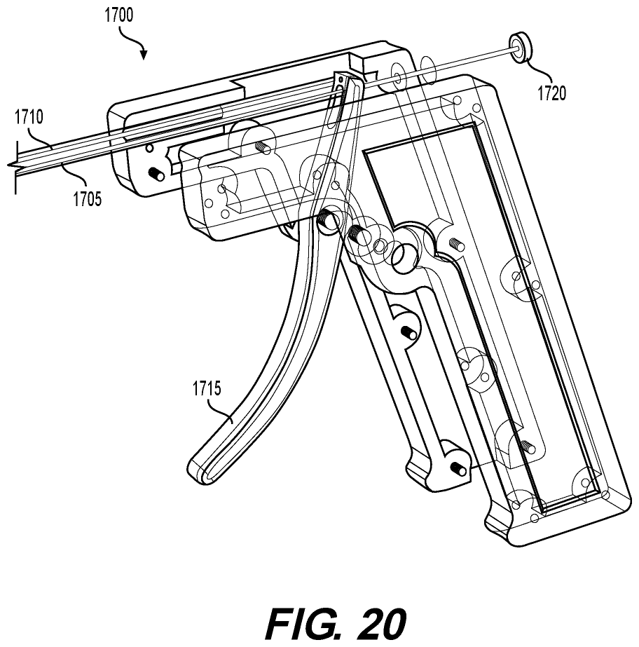

FIG. 20 is a schematic view of a proximal end of the insertion tool.

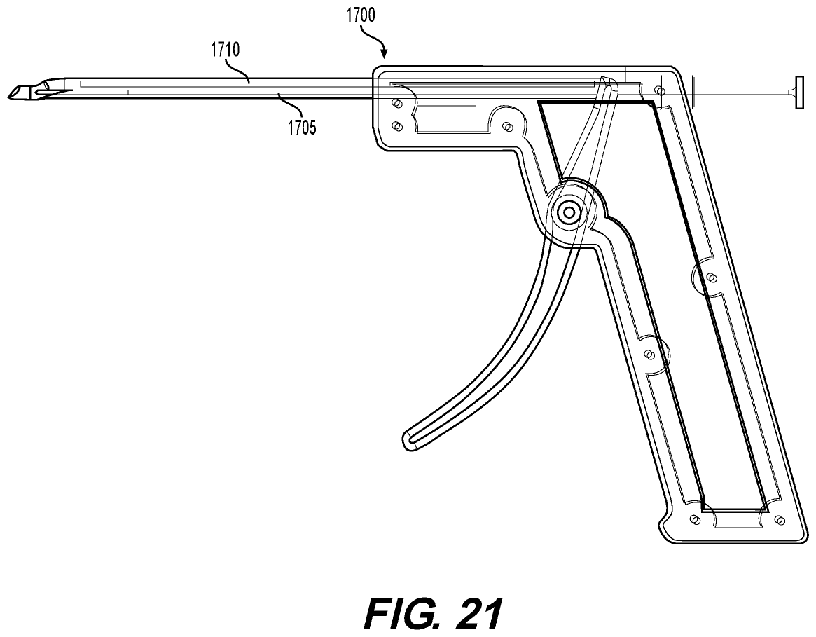

FIG. 21 is a schematic side view of the insertion tool. FIG. 21 shows tool 1700 with neither tissue piercing rod 1705 nor anchor delivery rod 1710 deployed.

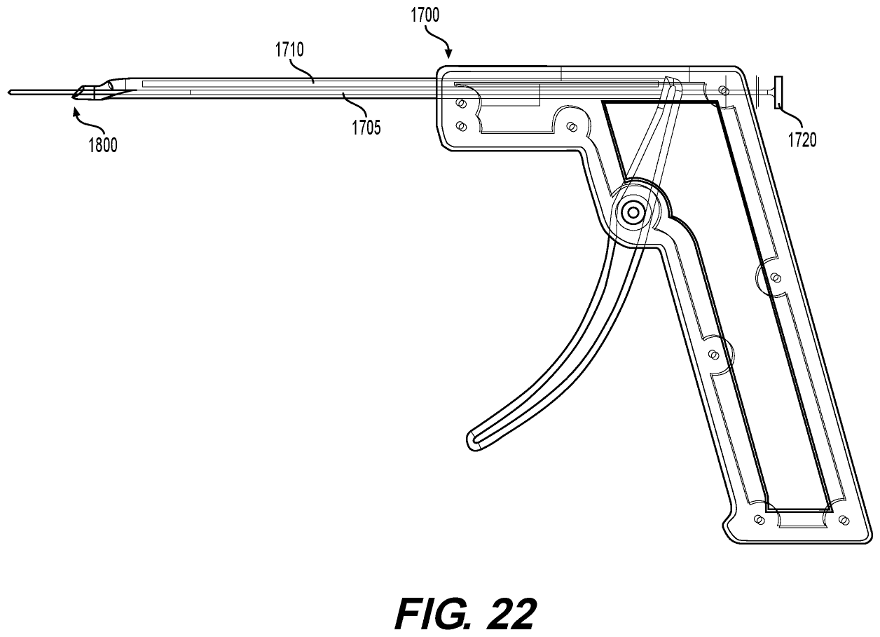

FIG. 22 is a schematic side view of the insertion tool with the tissue piercing rod deployed. FIG. 22 illustrates tissue piercing rod 1705 deployed beyond distal tip 1800. As shown in FIG. 22, plunger knob 1720 is translated to the left in order to deploy tissue piercing rod 1705.

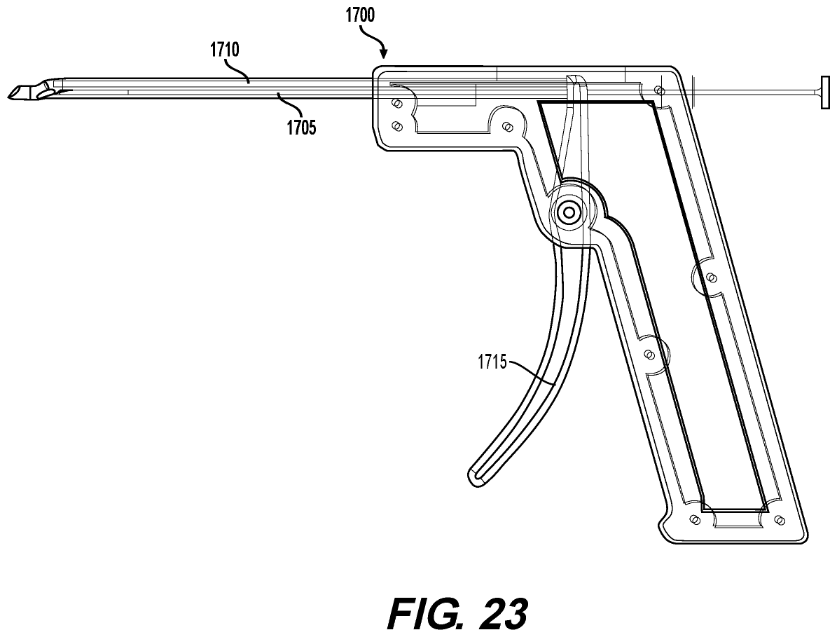

FIG. 23 is a schematic side view of the insertion tool with the anchor delivery rod partially deployed. As shown in FIG. 23, trigger handle 1715 is actuated, thus partially deploying anchor delivery rod 1710. In order to fully deploy anchor delivery rod 1710, trigger handle 1715 may be simply actuated further. Upon further deployment anchor delivery rod 1710 bends around a curve in a junction between the first lumen and the second lumen. In some embodiments, anchor delivery rod 1710 may be formed of nitinol, and thus, may have the flexibility to be delivered through this tortuous pathway in the inside of tool 1700.

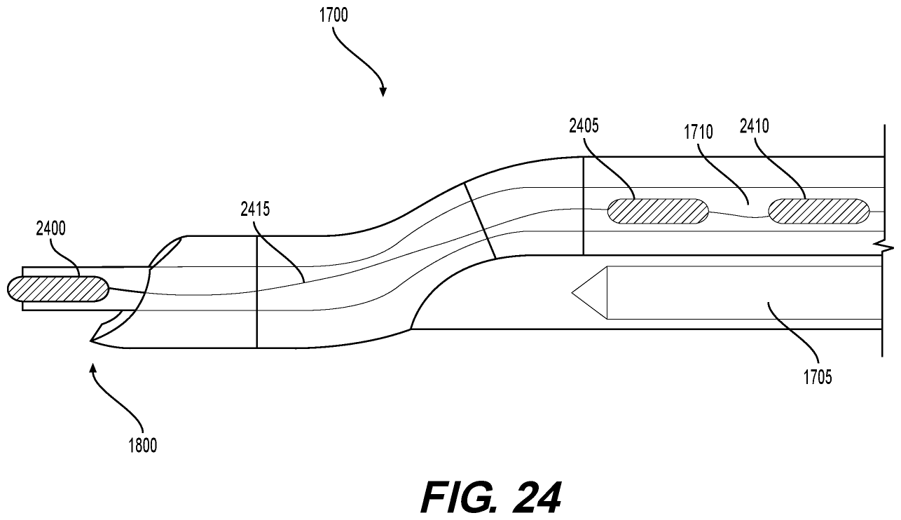

FIG. 24 is a schematic enlarged side view of the distal end of the insertion tool with the anchor delivery rod fully deployed and delivering a soft orthopedic anchor. As shown in FIG. 24, anchor delivery rod 1710 is fully deployed beyond distal tip 1800 of tool 1700. FIG. 24 shows a multi-anchor system being delivered by tool 1700. As shown in FIG. 24, a first anchor 2400 is currently being deployed. A second anchor 2405 and a third anchor 2410 are also queued up and ready for delivery following first anchor 2400. FIG. 24 also shows a suture strand 2415 connecting the three anchors. This anchor system may have any suitable configuration. For example, the daisy chain multi-anchor system described above may be delivered using insertion tool 1700.

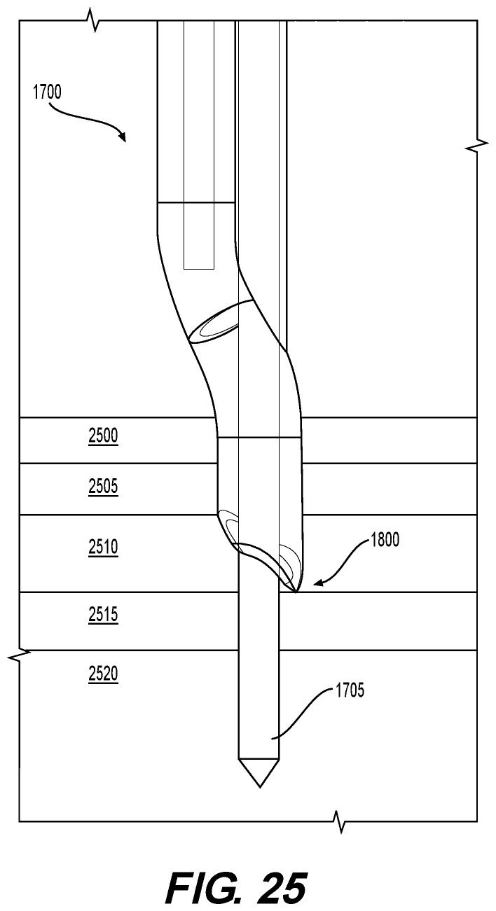

FIG. 25 is a schematic enlarged view of the insertion tool inserted into tissue and piercing bone with the tissue piercing rod. As shown in FIG. 25, distal tip 1800 of tool 1700 is inserted through a skin layer 2500, a tendon graft 2505, and a tendon 2510. FIG. 25 shows distal tip 1800 in contact with a cortical bone layer 2515. In order to pierce cortical bone layer 2515 and part of a cancellous bone layer 2520, tissue piercing rod 1705 is deployed. It will be noted that, although FIG. 25 depicts piercing of a bone layer, insertion tool 1700 may be used for piercing only soft tissues in some cases. For example, tool 1700 may be used to insert an anchor through only skin, a tendon graft, and the tendon itself.

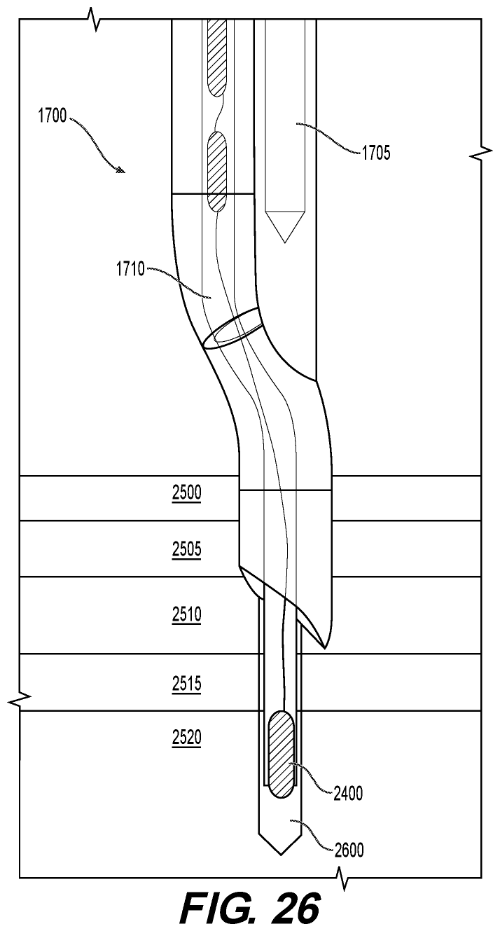

FIG. 26 is a schematic enlarged view of the insertion tool inserted into tissue and delivering a soft orthopedic anchor into the hole created by the tissue piercing rod. As shown in FIG. 26, tissue piercing rod 1705 has been retracted (i.e., by pulling on the plunger knob), leaving a hole 2600 through cortical bone layer 2515 and through part of cancellous bone layer 2520. FIG. 26 further shows first anchor 2400 being delivered into hole 2600 by anchor delivery rod 1710.

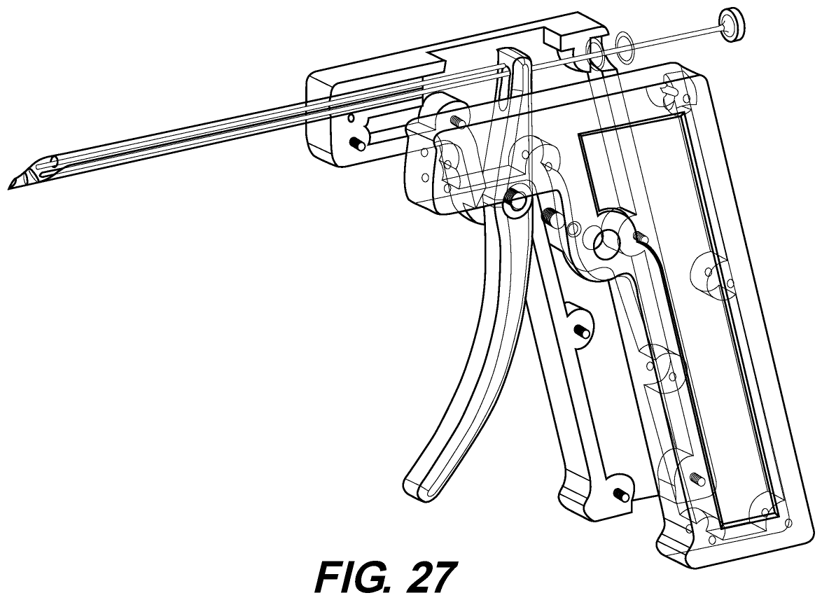

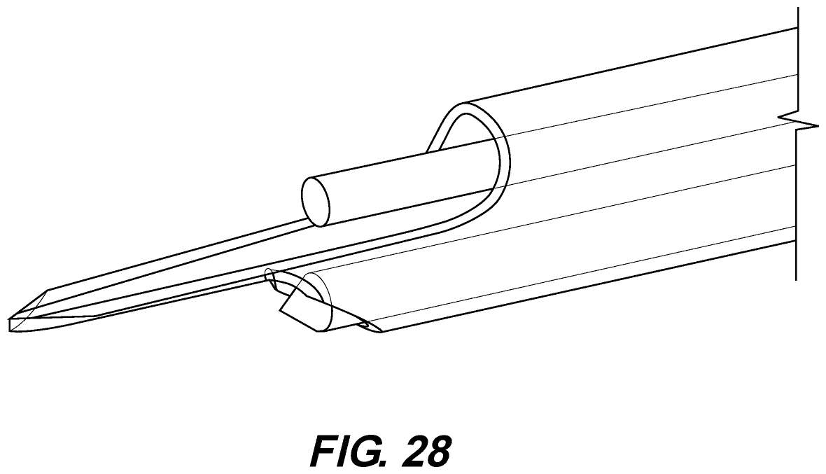

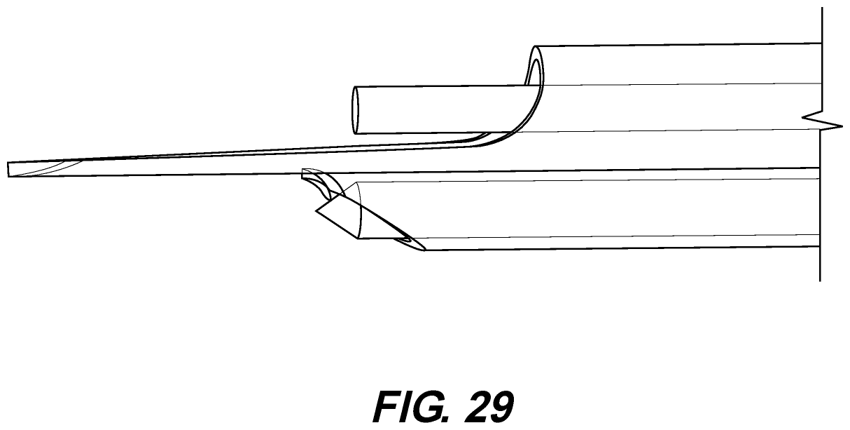

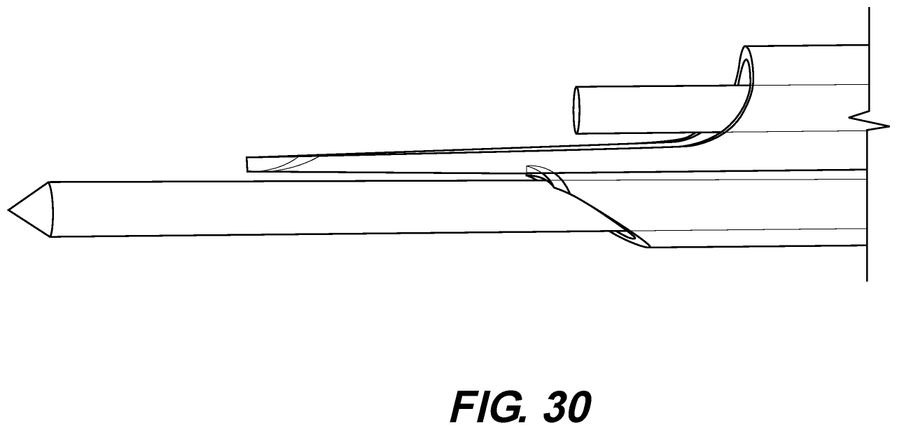

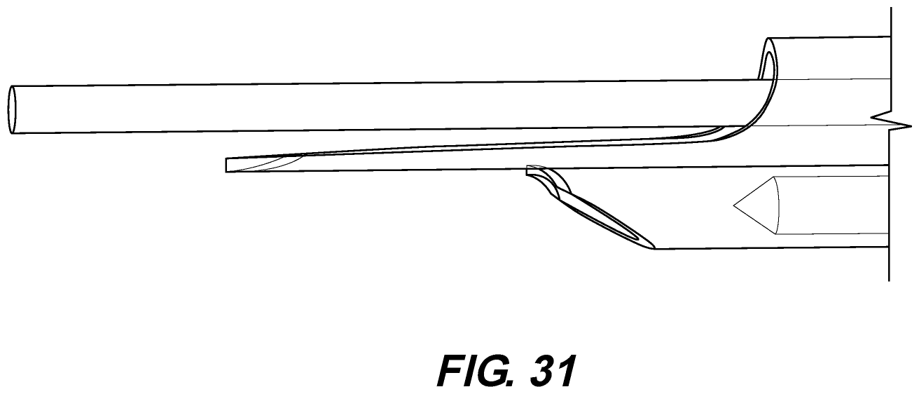

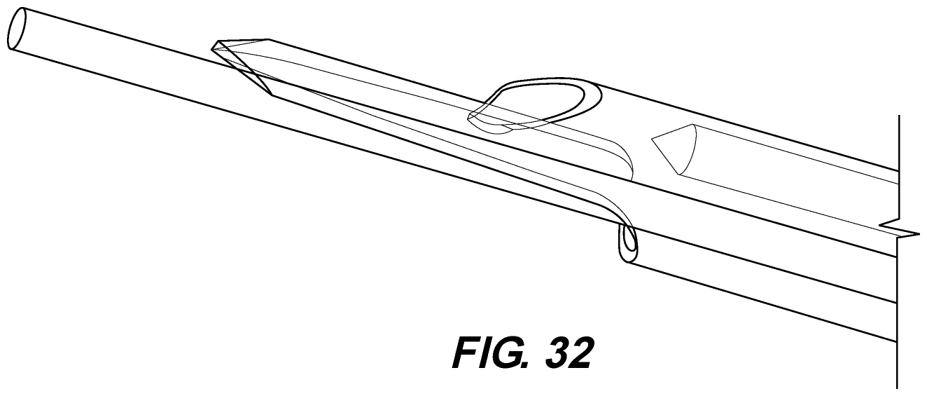

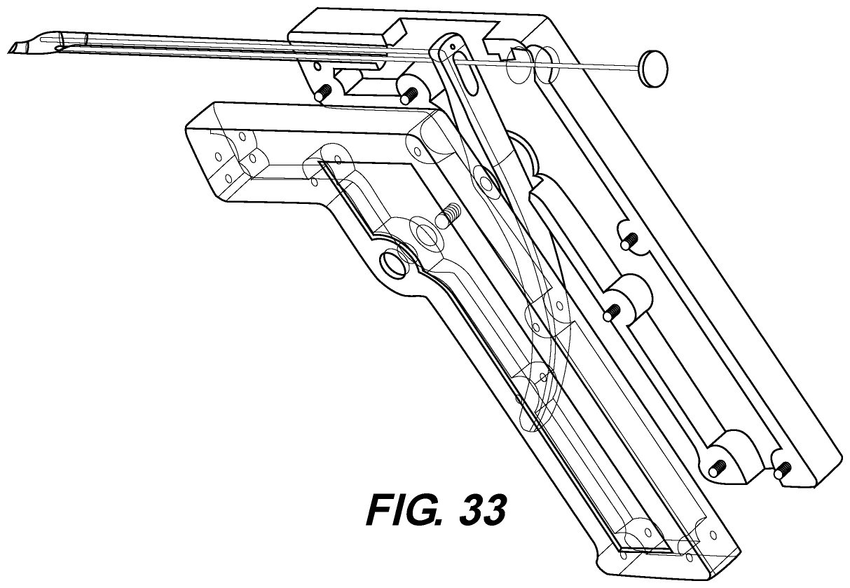

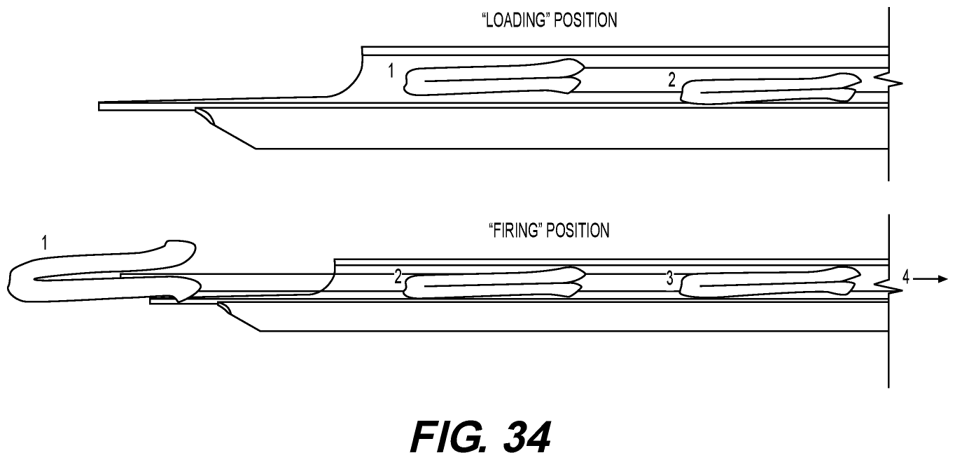

FIGS. 27-34 are schematic illustrations of an orthopedic anchor insertion tool according to another exemplary embodiment. The embodiment shown in FIGS. 27-34 includes two lumens, each having their own individual exits at the distal end of the insertion tool. The tool shown in FIGS. 27-34 may also be used to deliver multi-anchor systems in a similar manner as the embodiment shown in FIGS. 17-26. That is, the tool shown in FIGS. 27-34 may include a tissue piercing rod and an anchor delivery rod. The tool may utilize the same or substantially the same proximal structures. However, the distal tip of the tool may simply include two separate exits instead of one common opening for both lumens.

While various embodiments of the invention have been described, the description is intended to be exemplary, rather than limiting, and it will be apparent to those of ordinary skill in the art that many more embodiments and implementations are possible that are within the scope of the invention. Any element of any embodiment may be substituted for another element of any other embodiment or added to another embodiment except where specifically excluded. Accordingly, the invention is not to be restricted except in light of the attached claims and their equivalents. Also, various modifications and changes may be made within the scope of the attached claims.

1. An orthopedic anchor assembly, comprising:
an elongate, relatively soft orthopedic anchor; and
a suture strand extending from a first end of the orthopedic anchor through a sidewall of the orthopedic anchor and internally through the orthopedic anchor to a second end of the orthopedic anchor;
the suture strand further extending through the sidewall of the orthopedic anchor at the second end of the orthopedic anchor and extending back to the first end of the orthopedic anchor externally to the orthopedic anchor.

2. The orthopedic anchor assembly of claim 1, wherein the suture strand further extends into itself proximate the first end of the orthopedic anchor and extends coaxially within a length of the suture strand through the orthopedic anchor.

3. The orthopedic anchor assembly of claim 2, wherein the suture strand exits the length of suture strand proximate the second end of the orthopedic anchor.

4. The orthopedic anchor assembly of claim 1, wherein the suture strand is knotted proximate a location where the suture strand enters the sidewall of the orthopedic anchor proximate the first end of the orthopedic anchor.

5. The orthopedic anchor assembly of claim 1, wherein the suture strand is of braided construction.

6. The orthopedic anchor assembly of claim 1, wherein the suture strand is formed of a polymer.

7. The orthopedic anchor assembly of claim 1, wherein the suture strand is formed of high strength collagen.

8. The orthopedic anchor assembly of claim 1, wherein the suture strand is formed of a combination of high strength collagen and at least one other material.

9. The orthopedic anchor assembly of claim 8, wherein the at least one other material is ultra high molecular weight polyethylene (UHMWPE).

10. A multi-anchor system, comprising:
a first elongate, relatively soft orthopedic anchor;
a second elongate, relatively soft orthopedic anchor; and
a suture strand extending from a first end of the first orthopedic anchor through a sidewall of the first orthopedic anchor and internally through the first orthopedic anchor to a second end of the first orthopedic anchor;
the suture strand further extending through the sidewall of the first orthopedic anchor at the second end of the first orthopedic anchor;
the suture strand extending from the second end of the first orthopedic anchor to a first end of the second orthopedic anchor;
the suture strand further extending through the second orthopedic anchor to a second end of the second orthopedic anchor; and
the suture strand extending from the second end of the second orthopedic anchor back to the first end of the first orthopedic anchor.

11. The system of claim 10, wherein the suture strand further extends into itself proximate the first end of the first orthopedic anchor and extends coaxially within a length of the suture strand through the first orthopedic anchor and exiting the length of suture strand proximate the second end of the first orthopedic anchor.

12. The system of claim 10, further including at least a third elongate, relatively soft orthopedic anchor, wherein the suture strand extends through the third orthopedic anchor before returning to the first end of the first orthopedic anchor, such that the three orthopedic anchors are connected together with the suture strand.

13. The system of claim 10, wherein the suture strand is of braided construction.

14. The system of claim 10, wherein the suture strand is formed of high strength collagen.

15. The system of claim 10, wherein the suture strand is formed of a combination of high strength collagen and at least one other material.

16. The system of claim 15, wherein the at least one other material is ultra high molecular weight polyethylene (UHMWPE).

17. An insertion tool for an orthopedic anchor, comprising:
a tissue piercing rod received within a first lumen;
an anchor delivery rod received within a second lumen parallel to the first lumen; and
a distal tip including an exit lumen in communication with both the first lumen and the second lumen;
wherein the exit lumen is configured to alternately receive the tissue piercing rod and the anchor delivery rod.

18. A method of implanting an orthopedic anchor using the insertion tool of claim 17, the method comprising:
delivering the distal tip of the insertion tool to a surgical site and contacting a surface at the surgical site with the distal tip;
advancing the tissue piercing rod into tissue at the surgical site to create a hole in the tissue;
withdrawing the tissue piercing rod;
advancing the anchor delivery rod into the hole formed by the tissue piercing rod to deliver a relatively soft anchor into the tissue.

19. The method of claim 18, wherein the soft anchor is formed of a braided construction.

20. The method of claim 18, wherein the anchor is attached to a suture strand formed at least in part from collagen.
